(19) United States
(12) Patent Application Publication
(10) Pub. No.: US 2006/0025749 A1
(43) Pub. Date: Feb. 2, 2006

(54) Trocar-cannula complex, cannula and method for delivering fluids during minimally invasive surgery

(76) Inventor: Stephen P. Moenning, Punta Gorda, FL (US)

Correspondence Address: WOOD, HERRON & EVANS, LLP, 2700 CAREW TOWER, 441 VINE STREET, CINCINNATI, OH, 45202, US

(21) Appl. No.: 11/238,290

(22) Filed: Sep. 29, 2005

Related U.S. Application Data
(63) Continuation-in-part of application No. 10/786,647, filed on Feb. 25, 2004.
Application No. 10/786,647 is a continuation-in-part of application No. 09/934,399, filed on Aug. 21, 2001, now Pat. No. 6,695,815.
Application No. 09/934,399 is a continuation of application No. 09/511,100, filed on Feb. 23, 2000, now Pat. No. 6,302,873.

(30) Foreign Application Priority Data
Sep. 17, 2002 (WO) PCT/US02/29356

(51) Int. Cl. A61M 31/00

(52) U.S. Cl. 604/506; 604/161

(57) ABSTRACT

A fluid delivery cannula which provides an interface between an access point or port site in the body of a patient and a working channel which may receive tools or instruments used during surgical procedures which may be less invasive surgical procedures than traditional open procedures. The cannula allows introduction of fluid(s) into the port site and then into tissue generally at or near that location within the body of the patient. The fluid delivery cannula includes an expandable sleeve that may itself comprise a cannula through which a needle, or trocar assembly is inserted. At least one fluid passageway, is defined, for example, in either the expandable sleeve itself, or defined, by the combination of the needle or trocar assembly and the expandable sleeve. Visual identifiers are used with the fluid delivery cannula to visually distinguish the location of the fluid passageway relative to an adjacent area.

This application is a continuation-in-part of prior application Ser. No. 10/786,647, filed Feb. 25, 2004 (pending) which is a continuation of prior PCT Serial No. PCT/US02/29356 filed on Sep. 17, 2002 (expired) which claims the benefit of U.S. Provisional Patent Application Ser. No. 60/325,806, filed on Sep. 28, 2001 (now abandoned) and 60/341,032, filed on Dec. 12, 2001 (now abandoned), and which is a continuation-in-part of prior U.S. Ser. No. 09/934,399, filed on Aug. 21, 2001 (now U.S. Pat. No. 6,695,815) which is a continuation of prior U.S. Ser. No. 09/511,100 filed on Feb. 23, 2000 (now U.S. Pat. No. 6,302,873). The disclosures of each of these prior related applications are hereby fully incorporated by reference herein.

### FIELD OF THE INVENTION

This invention generally relates to cannulas and, more specifically, to cannulas used during surgery for allowing the introduction of instruments, such as laparoscopic, endoscopic, arthroscopic or other tools, during surgical procedures.

### BACKGROUND OF THE INVENTION

Various levels of less invasive surgery are popular alternatives to more traditional open surgical procedures. Such less invasive techniques are generally referred to herein as “minimally invasive,” however, some techniques are more invasive than others. Minimally invasive surgery generally results in less pain and shorter hospital stays for the patient. Also, performing a surgical procedure through less invasive techniques can be substantially less costly than more traditional surgical approaches.

Minimally invasive surgical techniques require access into the body of a patient through a small working channel of an apparatus, such as a trocar-cannula assembly, also known in various forms as a “trocar-cannula complex.” A relatively small access incision is made in the patient at the appropriate location on the patient to receive the trocar-cannula assembly. When the trocar-cannula assembly is combined with long, narrow instruments, the resulting assembly allows a surgeon to work at various locations inside the body through the small access incision or port site. For example, the location may be an abdominal cavity, joint cavity or other cavity or location in the body of the patient. This approach has resulted in the aforementioned clinical advantages and extensive health care cost savings.

Traditionally, the trocar-cannula complex has been configured with three parts. The first part is the top portion and is referred to in the medical industry as the hub. The hub defines the entrance to the trocar-cannula complex and also includes various seals and air insufflation components. The second part is the trocar, which is a long, narrow blade extendable through an inner cannula to allow smooth penetration into the body of the patient through the tissue layers. The third portion is an outer cannula which is a tubular member of the complex adapted to pass into the body cavity. The outer cannula provides an interface between the patient's tissue at the access incision or port site and the trocar assembly.

Minimally invasive surgery has grown in popularity in recent years and many new types of trocar-cannula products have been proposed and introduced to address different surgical needs and procedures. The various trocar-cannula complexes include reusable and disposable cannulas and trocars, as well as hybrid varieties that comprise combinations of reusable and disposable components of the trocar-cannula complexes. A complex which is a combination of reusable and disposable components is known as a resposable device. Such devices continue to improve surgical outcomes and economics.

Animal studies on cancer treatments involving the performance of minimally invasive surgery point to a growing body of evidence which supports the concept of delivering an irrigant to the port site after the surgical procedure. In these studies, the irrigants were delivered by a syringe and needle and included substances such as betadine, saline and lidocaine. These studies showed that irrigating the port site with such substances immediately after the surgical procedure beneficially resulted in a lower incidence of infection or less pain, depending on the irrigant. However, the technique also resulted in increased operative time and increased exposure of the surgical staff to needle sticks. In addition, the potential for contaminants to spread to the port site during the surgery has been well documented. Irrigation performed only at the end of the surgical procedure unfortunately cannot reduce patient exposure to contaminants during the procedure nor adequately reduce pain at port site.

In view of the above-mentioned drawbacks in the field, there is a need for more effective delivery of fluids to an access point or port in the body of a patient before, during, and/or after the performance of minimally invasive surgery. Such delivery of fluid(s) could assist in patient treatment, such as through the delivery of cancer treatment medication or other medication, as well as reduction of port site contamination and infection, and further reduction of post-operative pain as compared to injection at the end of the case. Other uses of the invention may be made in connection with delivering any desired fluid substance to a patient.

### SUMMARY OF THE INVENTION

One form of the present invention is a method for administering fluid directly to a port site located in a section of tissue. A radially expanding tubular structure is introduced into the port site. The radially expandable tubular structure includes an outer surface adapted to interface with the port site and defines a lumen. The fluid is delivered to the port site via at least one fluid passageway in fluid communication with the outer surface. The at least one fluid passageway includes a portion at least defined in part by the radially expandable tubular structure.

Another form of the invention includes a method of administering fluid directly to a port site located in a section of tissue. The method includes placing an insert into a lumen defined in the radially expandable tubular structure having an outer surface adapted to interface with the port site. The radially expandable tubular structure and insert is introduced into the port site. The fluid is delivered to the port site via at least one fluid passageway in fluid communication with the outer surface. The at least one fluid passageway includes a portion at least defined in part by the radially expandable tubular structure.

In another form, the invention includes an apparatus to administer fluid into a port site formed in an area of tissue from a location outside of the port site via at least one fluid passageway. The apparatus includes a radially expandable tubular structure defining a lumen. The radially expandable tubular structure includes an outer surface constructed and arranged to interface with tissue. The at least one fluid passageway is in fluid communication with the outer surface and includes a portion at least defined in part by the radially expandable tubular structure. The apparatus also includes an insert passing into the lumen.

A further form of the invention is a kit for administering fluid into a port site formed in a section of tissue from a location outside of the port site. The kit includes a radially expandable tubular structure defining a lumen. The radially expandable tubular structure includes an outer surface adapted to interface with the port site and a distal end. The kit further includes a needle for insertion into the lumen that assists with implanting the radially expandable tubular structure. A cannula is included having an external diameter greater than the diameter of the lumen of the tubular structure in its unexpanded state. Insertion of the cannula into the lumen radially expands the radially expandable tubular structure. A fluid pathway is in fluid communication with a location outside of the port site and at least one of the outer surface and the distal end.

Another form of the invention is a method for directing the application of fluid into a port site formed in a patient. A fluid delivery device defining at least one fluid passageway in fluid communication with the port site is introduced into the port site. A visual identifier located on the fluid delivery device is observed to visually distinguish the location of the at least one fluid passageway relative to an adjacent area of the fluid delivery device.

Various objects, advantages and features of the invention will become more readily apparent to those of ordinary skill upon review of the following detailed description of the illustrative embodiments taken in conjunction with the accompanying drawings.

### BRIEF DESCRIPTION OF THE DRAWINGS

FIG. 1 is a perspective view showing a trocar-fluid delivery cannula complex constructed in accordance with the invention and being used during a minimally invasive surgical procedure.

FIG. 2 is a cross sectional view taken generally along the longitudinal axis of the trocar-fluid delivery cannula complex of FIG. 1 for showing the irrigant flow path.

FIG. 3 is an enlarged cross sectional view similar to FIG. 2, but more clearly showing the flow path for the delivery of fluid through the cannula.

FIG. 4 is a cross sectional view taken along line 4-4 of FIG. 2.

FIG. 5 is a plan view of the fluid delivery cannula with the outer layer or sheath removed for clarity.

FIG. 6 is a plan view of another embodiment in which the fluid delivery cannula is integrally formed with a portion of a trocar hub.

FIG. 7 is a cross sectional view taken along line 7-7 of FIG. 6.

FIG. 8 is a longitudinal cross sectional view similar to FIG. 2, but illustrating an alternative embodiment of the invention incorporating an expandable fluid delivery sleeve.

FIG. 9 is a perspective view of another alternative embodiment of an expandable fluid delivery sleeve or cannula.

FIG. 10 is a cross sectional view taken along line 10-10 of FIG. 9.

FIG. 11 is an enlarged perspective view of the distal end of another expandable fluid delivery sleeve or cannula.

FIG. 12A is an elevational view illustrating inserting a needle into the expandable fluid delivery sleeve of FIG. 9.

FIG. 12B is a elevational view illustrating introducing the expandable fluid delivery sleeve of FIG. 9 into a port site and administering fluid.

FIG. 12C is an elevational view illustrating removing the needle from the expandable fluid delivery sleeve of FIG. 9.

FIG. 12D is an elevational view illustrating a trocar cannula assembly being inserted into the implanted expandable fluid delivery sleeve of FIG. 9.

FIG. 12E is an elevational view illustrating that fluid can optionally be administered after the trocar cannula assembly has been inserted into the expandable fluid delivery sleeve.

FIG. 13A is a cross sectional view of the combination of the expandable fluid delivery sleeve of FIG. 9 and the needle illustrated in FIG. 12B highlighting a fluid pathway through the sleeve.

FIG. 13B is a cross sectional view of the combination of the expandable fluid delivery sleeve of FIG. 9 and the trocar cannula assembly illustrated in FIG. 12E highlighting a fluid pathway through the sleeve.

FIG. 14 is a cross sectional view of the combination of the expandable fluid delivery sleeve and needle illustrated in FIG. 12B highlighting a fluid pathway through the area defined between the needle and the sleeve.

FIG. 15 is a partial cross section view through the expandable fluid delivery sleeve of FIG. 9 highlighting a fluid pathway through the area between the trocar cannula assembly and the sleeve.

FIG. 16 is an enlarged cross sectional view of an alternative embodiment of the insert of FIG. 12A illustrating a fluid passageway through a lumen in the needle.

FIG. 17 is a perspective view of the expandable fluid delivery sleeve and needle of FIG. 12A illustrating a color coding technique to highlight a zone containing perforations on the sleeve.

### DETAILED DESCRIPTION OF THE ILLUSTRATIVE EMBODIMENTS

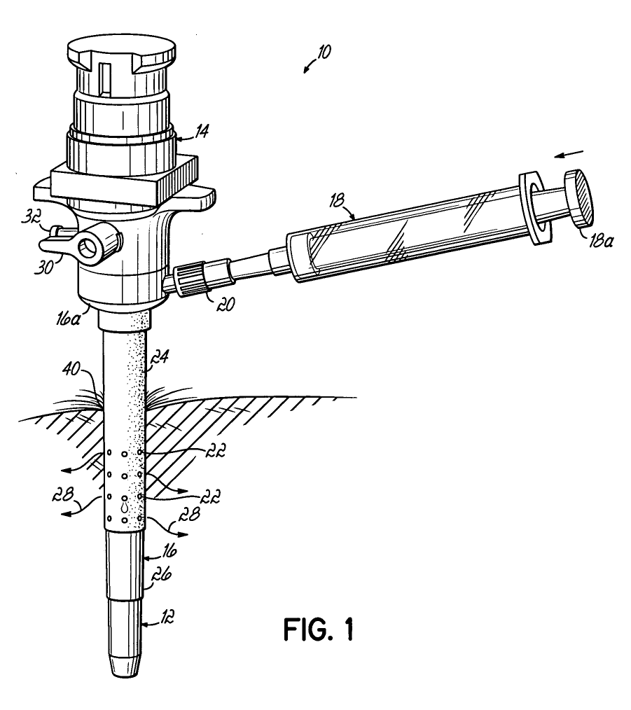

FIG. 1 illustrates a trocar-fluid delivery cannula complex 10 constructed in accordance with one preferred embodiment of the invention. Complex 10 includes a trocar assembly 12 which may include a conventional hub assembly 14. Representative trocar assemblies are shown and described in previous patents, such as my previous U.S. Pat. Nos. 6,063,060; 6,039,725; 5,865,817; and 5,865,809, the disclosures of which are hereby fully incorporated by reference herein. In accordance with the invention, a cannula 16 is positioned on the outside of trocar assembly 12 and includes a base portion 16a. A syringe 18 couples to base portion 16a of cannula 16 through a fluid coupling, such as a standard luer connector assembly 20. A plunger 18a of syringe 18 is used to manually inject a fluid into base portion 16a of cannula 16. An outer layer or sheath 24, preferably formed of PTFE (Teflon®), is secured to the outer surface of an inner tube 26 of cannula 16 and includes apertures 22. In the preferred embodiment, sheath 24 is a tube which is heat shrunk onto inner tube 26 but it may take other forms and may be secured in other ways. As will be described below, cannula 16 includes appropriate fluid passages communicating with an inlet passage in base portion 16a to allow the fluid to be dispensed through apertures 22 as shown by arrows 28. Hub assembly 14 further includes an insufflation valve 30 and a gas inlet 32 for receiving a pressurized gas, such as CO₂.

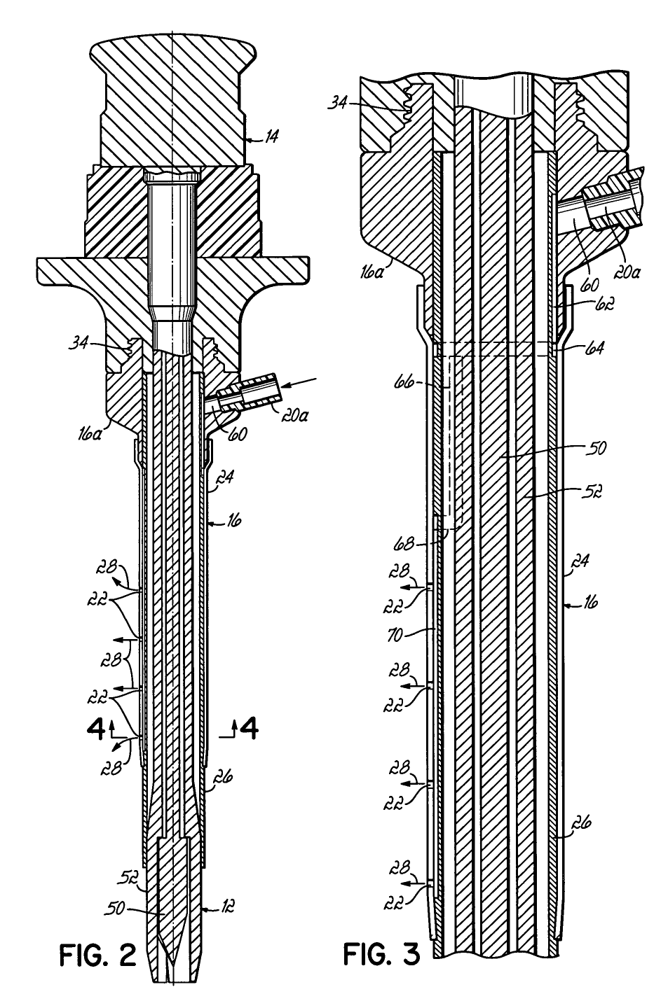

As further shown in FIGS. 2 and 3, base portion 16a of cannula 16 is threaded onto hub assembly 14 by threads 34. Thus, cannula 16 may be easily coupled to and decoupled from hub assembly 14. In the preferred embodiment, cannula 16 is disposable, however, it also may be manufactured as a reusable device intended to be sterilized between uses. Trocar assembly 12 more specifically comprises a trocar 50 received by a protective shield 52. It will be appreciated that other instruments and tools may be inserted through the working channels formed by either irrigating cannula 16 or other tubular member(s) positioned within cannula 16. This includes many other configurations of trocars or trocar assemblies as generally recognized in the art.

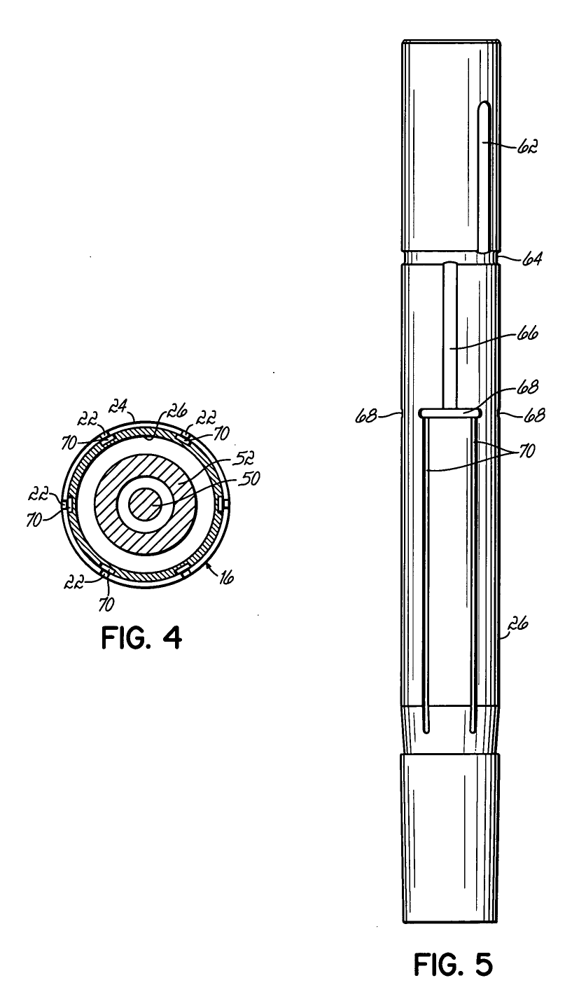

More specifically referring to FIGS. 3-5, irrigation fluids are introduced through luer connector 20a (FIG. 3) into fluid inlet 60 and groove or channel 62 formed in inner tube 26 of cannula 16. Groove 62 communicates with an annular, circumferential groove 64 and groove 64 communicates with three separate longitudinal grooves 66 which are spaced in 120° increments about inner tube 26. Grooves 66 respectively communicate with three partially annular grooves 68 which, in turn, each communicate with two longitudinal grooves 70. Longitudinal grooves 70 communicate with apertures 22 in sheath 24 and apertures 22 thereby dispense the fluid at the port site 40 or, if cannula 16 is appropriately inserted and positioned, elsewhere within the patient.

As mentioned above, the outer sheath 24 of the cannula 16 is preferably formed of PTFE and, more preferably, the outer sheath 24 is transparent or at least translucent. In addition, the area of sheath 24 containing apertures 22 may be formed with a distinct color, texture or other visually identifiable indicia which allows the surgeon to accurately position the apertures 22 with respect to the tissue to be infused with irrigation fluid. The various grooves in the outside surface of the inner tube 26 may be substituted with one or more passages within the walls of the inner tube 26 and may be of any suitable configuration and shape so long as the function of delivering fluid through the wall of the cannula 16 is facilitated by the configuration. The outer wall or sheath is a heat shrinkable material, such as an elastomeric material, however, this may also be substituted by other components or even eliminated, for example, if the passages and apertures are in the wall of an integrally formed cannula or if another fluid delivery structure is carried on the outer cannula. The inner tube in the preferred embodiment is preferably formed from aluminum with the various grooves in its outer surface being machined, however, it may instead be formed of other materials, such as plastic materials, and formed by other techniques such as molding. The preferred embodiment is especially advantageous in that it is simple to manufacture and the outer sheath forms a seal at the upper and lower ends of the inner tube while, at the same time, defining walls of the internal passages formed by the various grooves.

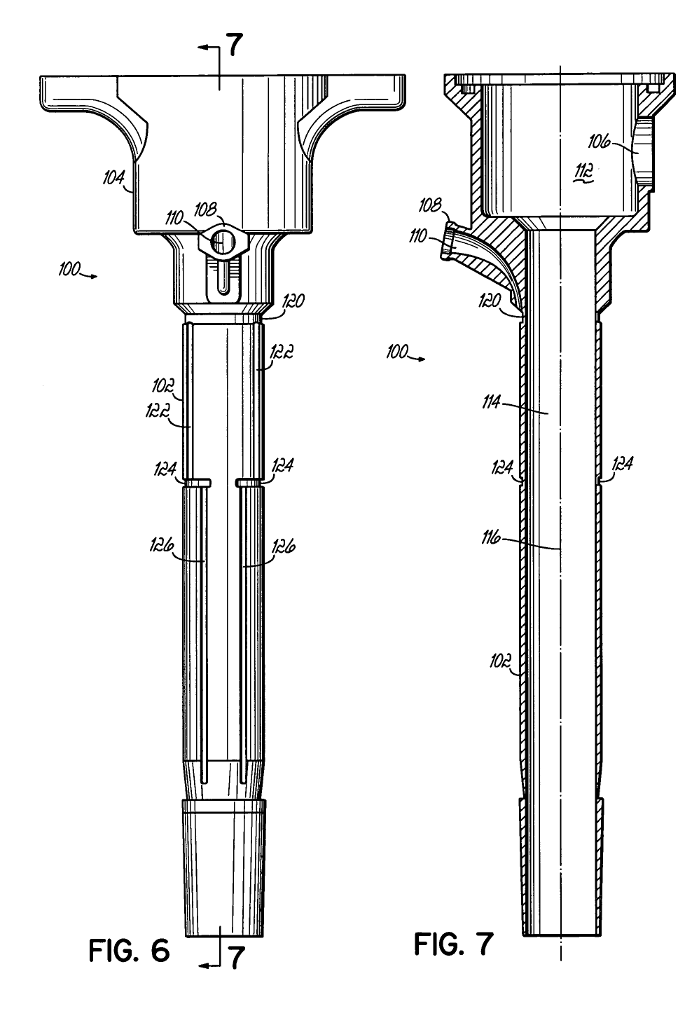

FIGS. 6 and 7 illustrate a second illustrative embodiment of the invention comprising a fluid delivery cannula 100 which includes an irrigating portion 102 and a hub or housing portion 104 formed in one piece. For example, the entire structure shown in FIGS. 6 and 7 may be molded from a polymeric material, such as conventional medical grade polymers, using Mu-cell technology or other appropriate molding techniques. In FIGS. 6 and 7, the outer layer or sheath containing the one or more perforations has been removed for clarity. Housing portion 104 includes a port 106 for receiving valving and gas input components as are known in the art. A fluid input 108 is formed on cannula 100 and communicates with a passage 110 for the introduction of the necessary or desired fluids to irrigation portion 102. A space 112 is provided for the necessary valving, sealing components, etc., typically used in trocar hubs. A lumen 114 extends along an axis 116 for receiving the trocar (not shown) and other working instruments. A system of fluid delivery passages is formed on the outside surface of irrigation portion 102 in the same illustrative pattern as discussed with respect to the first embodiment. This includes an annular groove 120 which communicates with passage 110 and delivers the fluid to three separate longitudinal passages 122 positioned at 120° increments around the outside surface of irrigation portion 102 relative to axis 116. Grooves 122 communicate with respective partially annular grooves 124. Again, while only two grooves 124 are shown in the drawings, a total of three grooves are formed in the outer surface of irrigation portion 102 positioned at 120° increments about axis 116. Each partially annular groove 124 communicates with two separate longitudinal grooves 126. Although only two grooves 126 are shown in FIG. 6, it will be appreciated that a total of six such grooves are formed in the outer surface of irrigation portion 102 in this particular embodiment. As in the first embodiment, grooves 126 communicate the fluid to perforations in the outer sheath (not shown) which then deliver the fluid to the patient. The outer sheath, as in the first embodiment, is preferably heat shrunk onto irrigation portion 102 so as to seal all of the grooves in the same manner as shown, for example, in FIGS. 2 and 3 of the first embodiment. As mentioned above, it will be appreciated that many other configurations of fluid delivery passages may be utilized in the cannula within the spirit and scope of this invention.

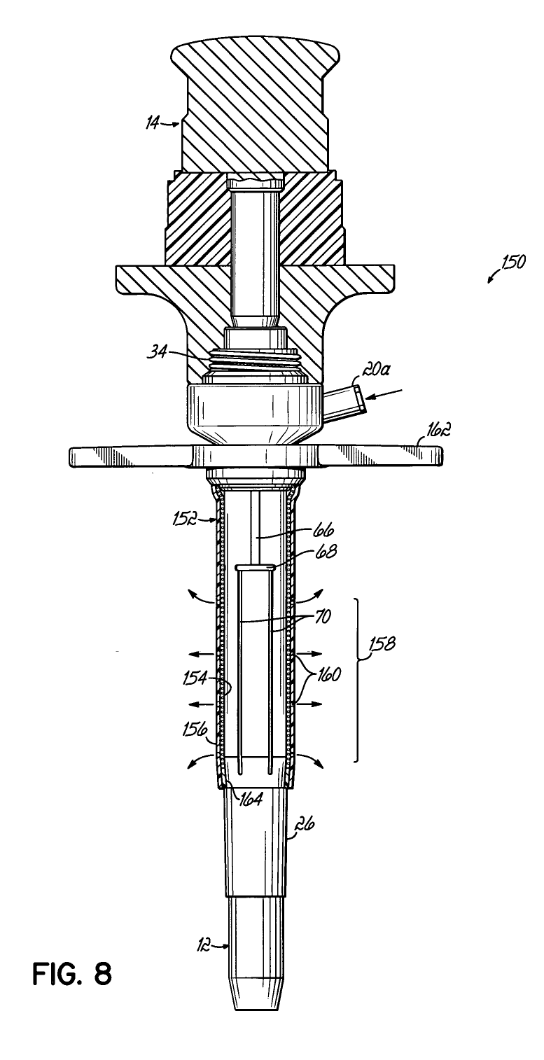

In FIG. 8, like reference numerals refer to like elements of structure between the two embodiments. In the alternative trocar-cannula complex 150 of FIG. 8, the outer sleeve or layer 24 (not shown) which was affixed to the grooved cannula 26 has been removed and replaced by an expandable sleeve 152. Expandable sleeve 152 may be a layered construction including a mesh layer 154 and an outer elastomeric layer 156. Layer 156 is uniformly perforated about its entire periphery, such as in a circumferential zone 158 as shown in FIG. 8, so that at least some of the perforations 160 line up with the longitudinal grooves 70 of the cannula 26. Thus, fluid is delivered through input 20a and into grooves 66, 68, 70 as described previously with respect to the first embodiment and this fluid is transferred through the expandable inner mesh layer 154 and expandable outer elastomeric layer 156 containing perforations 160. It will be appreciated that many other forms than the layered mesh construction shown may be used in place of the expandable sleeve 152 shown in FIG. 8. FIG. 8 illustrates the use of the expandable sleeve 152 in connection with a 10 mm trocar assembly, however, in accordance with this aspect of the invention, the expandable fluid delivery sleeve 152 may alternatively be used with other trocars having larger or smaller diameters. A rigid handle portion 162 is provided at the proximal end of sleeve 152 to allow application and removal of sleeve 152 to and from trocar 12. In order to seal the distal end of the expandable sleeve, a seal 164 may be provided distally of the mesh layer 154 as generally illustrated in FIG. 8. Alternatively, this seal 164 may be eliminated and the mesh layer 154 could then allow additional fluid to be delivered from the distal end of the sleeve 152.

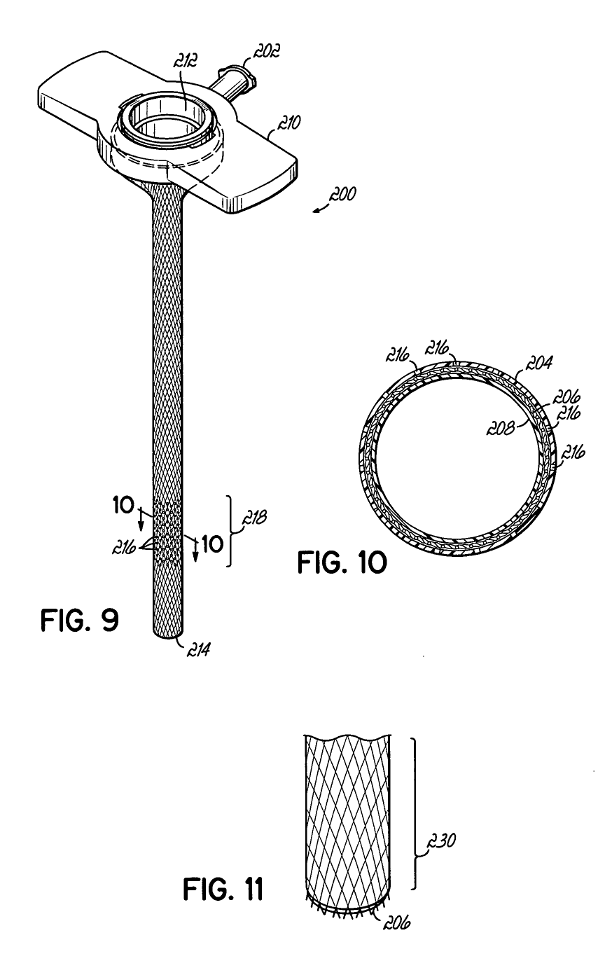

FIGS. 9 and 10 illustrate another embodiment of an expandable fluid delivery sleeve 200 which does not need the separate cannula 26 (FIG. 8) for fluid delivery as in the embodiment of FIG. 8. Instead, this sleeve 200 is formed in a manner allowing fluid delivery to take place via an input 202 and sleeve 200 alone. Sleeve 200 is formed of a layered construction including an outer perforated layer 204, an intermediate mesh layer 206, and an inner layer 208. Each layer 204, 206, 208 is expandable such that sleeve 200 may be used effectively on trocars having different diameters. The intermediate mesh layer 206 allows fluid to travel through the interstices therein from an appropriate fluid passageway extending through input 202 and an upper handle portion 210. Alternatively, other types of fluid passages may be utilized. A trocar (not shown) is inserted through the bore 212 at the proximal end such that it extends through the distal end 214 of the expandable sleeve 200. Perforations 216 are preferably formed in a desired zone 218 of sleeve 200 generally as described with respect to the previous embodiments. This zone 218 may be formed of a different color or in any other manner which indicates the positioning of the perforations to the doctor during the surgical procedure. Although not shown in FIGS. 9 and 10, this sleeve 200 may also have a seal at the distal end 214 to prevent fluid from leaking out the distal end 214.

As exemplified in FIG. 11, a distal end 230 of the expandable sleeves may be formed so as to allow fluid delivery to take place directly at the distal end. This aspect is shown in FIG. 11 schematically by indicating that the intermediate mesh layer 206 extends slightly beyond the other layers or is otherwise unsealed and, therefore, the fluid pathway through the mesh material 206 remains unblocked at the distal end 230. This general aspect of fluid delivery from the distal end 230 may be used alone or in conjunction with fluid delivery from surface perforations as previously described.

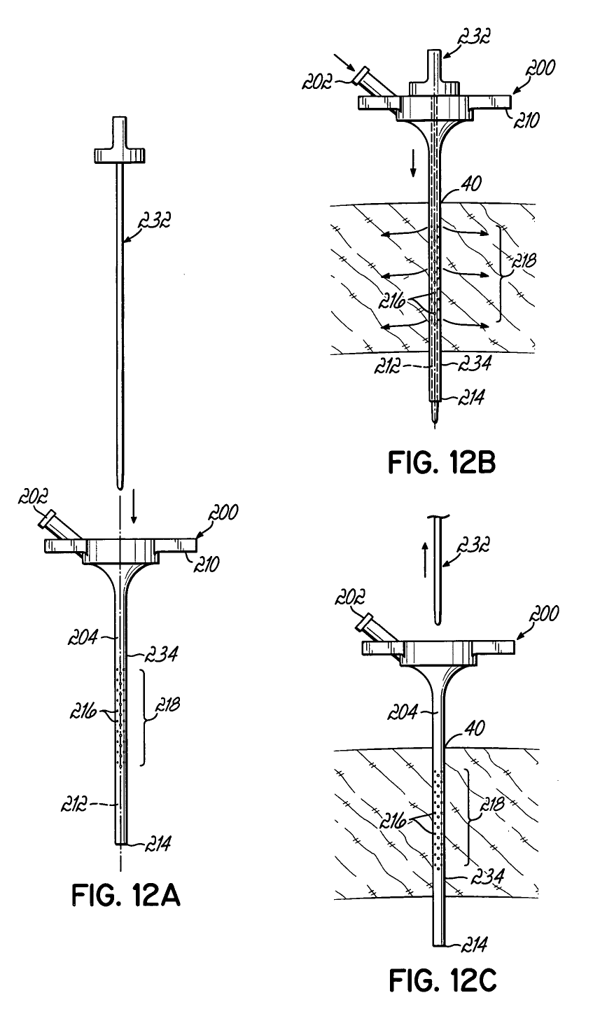

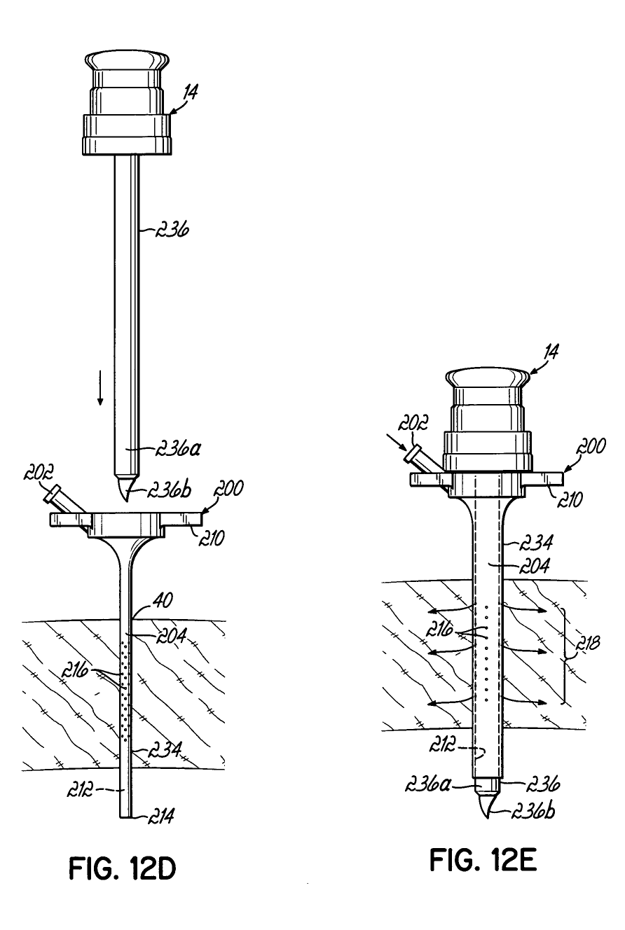

Referring now to FIGS. 12A through 12E, a method for administering fluid directly to a port site using the expandable fluid delivery sleeve 200 is illustrated. Identical reference characters refer to identical aspects of the embodiments that have already been described. This method is illustrated to apply to a laparoscopic surgery, however, it can apply to other medical procedures. Initially, the expandable fluid delivery sleeve 200 is introduced into the port site 40. FIG. 12A illustrates placing a needle or other insert 232 into the bore or lumen 212 of the expandable fluid delivery sleeve 200. The form of the needle 232 can vary in alternate embodiments. For example, the needle 232 could be the Step™ Insulflation/Access Needle, 14 gauge size available from Autosuture located in Norwalk, Conn. This needle 232 is compatible with Versastep™ Plus Access systems. The Step™ needle is a blunt headed needle and does not form the incision. However, those skilled in the art recognize that in other embodiments the needle 232 can be designed to form the incision to introduce the expandable fluid delivery sleeve 200 into the patient. The Step™ needle is composed of a metallic material, however, in other embodiments other materials can be used. Moreover, the gauge of the needle 232 can vary from embodiment to embodiment.

FIG. 12B illustrates the expandable fluid delivery sleeve 200 inside the port site 40. The combination of the needle 232 and the expandable fluid delivery sleeve 200 is implanted into the port site 40 formed in the patient. As FIG. 12B illustrates, fluid is then delivered to the port site 40 via a fluid passageway that connects an area outside of the port site 40, such as, but not limited to, the fluid input 202 to the outer surface 234 of the outer perforated layer 204.

Note that the fluid can be applied at different times in different embodiments. For example, FIG. 12E described herein below, illustrates delivering the fluid after the needle 232 has been removed and a trocar and cannula assembly 236 has been inserted. Alternatively, the fluid could be delivered after the needle 232 has been removed but before the trocar cannula assembly 236 has been inserted. Accordingly, those skilled in the art recognize that the fluid could be delivered any time the expandable fluid delivery sleeve 200 has been introduced into the port site 40 in differing embodiments.

Referring now to FIG. 12C, the needle 232 is illustrated as being removed from the expandable fluid delivery sleeve 200. At this point in the process, the expandable fluid delivery sleeve 200 is now lodged in the port site 40 but has not yet been dilated to allow access by the medical professional.

FIG. 12D illustrates a trocar and cannula assembly or other second insert 236 being inserted into expandable fluid delivery sleeve 200. Insertion of the trocar cannula assembly 236 achieves two purposes. First, the cannula 236a is easily slid into the expandable fluid delivery sleeve 200 because of the shape of the trocar 236b. Second, the cannula 236a radially expands the expandable fluid delivery sleeve 200 and provides a working opening or channel for the medical professional.

Referring now to FIG. 12E, an alternative step is illustrated. In this step, fluid is delivered to the port site 40 using a fluid passageway that is in communication between the inlet 202 and outer surface 234 after the trocar and cannula assembly 236 has already been inserted. A fluid is applied through the desired zone 218 because of the perforations 216 formed in the outer surface 234. In this embodiment, the fluid passes through outer surface 234 using perforations 216, however, in other embodiments, alternative methods and fluid paths may be used.

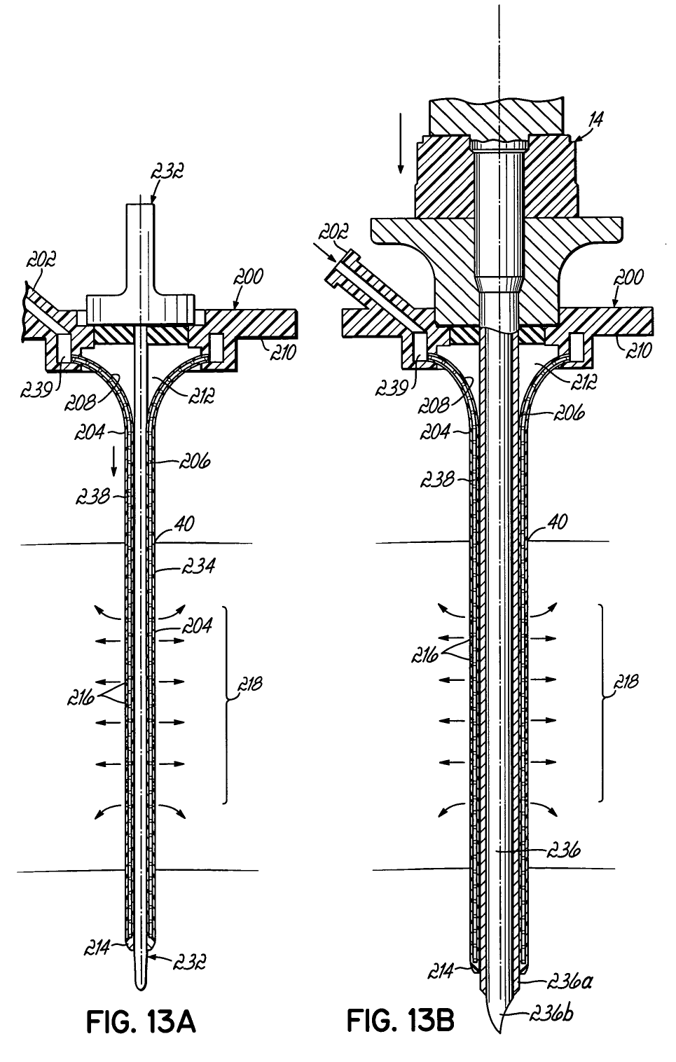

FIGS. 13A and 13B illustrate one technique for delivering fluid from the inlet 202 to the outer surface 234. In these embodiments, the inlet 202 is a port which is in fluid communication with a channel 238 that is defined between the outer layer 204 of the expandable fluid delivery sleeve 200 and the bore or lumen 212 of the expandable fluid delivery sleeve 200. The channel 238 begins at the inlet 202 and communicates with an annular groove or path 239 defined in the upper handle portion 210. The channel 238 then proceeds down through the area defined between the outer surface 234 of the expandable fluid delivery sleeve 200 and the bore or lumen 212 defined therein. The bore or lumen 212 of the expandable fluid delivery sleeve 200 is illustrated in this embodiment as being occupied by the needle 232. This channel 238 could be filled with a mesh layer 206 as illustrated in FIG. 10 or could be simply an open layer with nothing contained therein. Alternatively, the channel 238 can be defined in alternate manners besides layers. In some embodiments, the channel 238 is sealed at the distal end 214 to ensure that the fluid passes through perforations 216 defined in the outer layer 204. In other embodiments, a sufficient seal is formed when the expandable fluid delivery sleeve 200 is passed into the port site 40, therefore obviating the need for the channel 238 to be closed at the distal end 214. FIG. 13B illustrates a very similar cross sectional view to FIG. 13B. The difference between the figures lies in the fact that a trocar and cannula assembly 236 is inserted into the expandable fluid delivery sleeve 200 in FIG. 13B. As in FIG. 13A, the channel 238 delivers the fluid from inlet 202 to the outer surface 234 of the expandable fluid delivery sleeve 200. As with the embodiment illustrated in FIG. 13A, the distal end 214 of the expandable fluid delivery sleeve 200 can be open or closed.

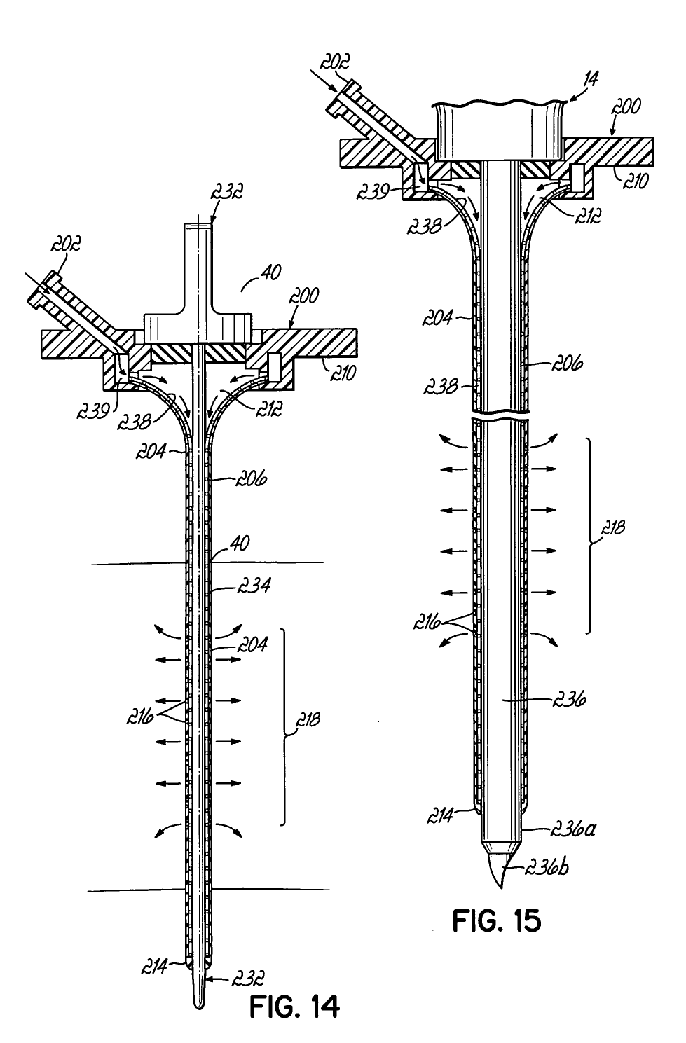

Referring now to FIG. 14, an alternative technique for delivering the fluid to the outer surface 234 of the expandable fluid delivery sleeve 200 is illustrated. Initially, the fluid enters into the expandable fluid delivery sleeve 200 through inlet 202. In the illustrated embodiment, inlet 202 is a port designed to receive a syringe, however, in other embodiments it could be an alternative passageway enabling fluid to pass into the expandable fluid delivery sleeve 200. Moreover, in other embodiments, the fluid is delivered using a suitable medical pump or other device. In this embodiment, the fluid enters into inlet 202 and then passes down into the annular groove 239 and then into the area defined between the bore or lumen 212 of the expandable fluid delivery sleeve 200 and the needle 232. The outer layer 204 of the expandable fluid delivery sleeve 200 has perforations 216 defined thereto that allow the fluid to reach the outer surface 234. Again, as in FIGS. 13A and 13B, the distal end 214 of the expandable fluid delivery sleeve 200 can be closed or open. FIG. 15 illustrates the same technique except with a trocar and cannula assembly 236 inserted into the expandable fluid delivery sleeve 200.

The bore or lumen 212 of the expandable fluid delivery sleeve 200 includes the mesh layer 206 in the embodiment illustrated in FIG. 15, however, in other embodiments the mesh layer 206 is not included in the lumen 212. Moreover, in some embodiments, an additional cannula or any other structure defining a fluid flow path can be used and positioned anywhere between an inner surface or structure defining lumen 212 and the outer surface or structure of the structure positioned therein.

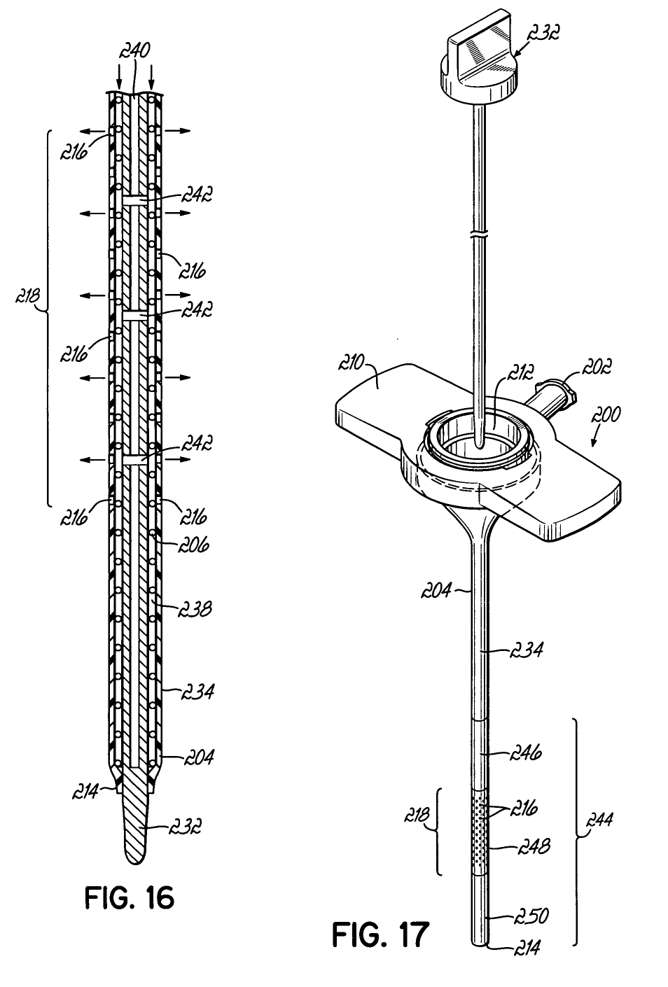

FIG. 16 illustrates another technique for delivering a fluid to the outer surface 234 of the expandable fluid delivery sleeve 200. In this embodiment, the needle 232 is shown placed into sleeve 200 and the needle 232 has a lumen 240 defined therein. A fluid can therefore pass from the inlet 202 at the top, or along another portion of the needle 232, and down through the lumen 240. The fluid passing through lumen 240 flows through cross bores 242 that deliver the fluid outside of the needle 232 and into the lumen 212 of the expandable fluid delivery sleeve 200. From there, the fluid flows out the perforations 216 to the outer surface 234 of the expandable fluid delivery sleeve 200. Those skilled in the art will recognize that instead of forming a lumen or fluid path in a needle one could be formed in a trocar and cannula assembly. A lumen or fluid path can be defined through the trocar with cross bores through the trocar. In addition, cross bores through the cannula could be formed as well to provide a fluid path. Other fluid paths could be used as well.

Referring now to FIG. 17, a color coding system is illustrated. FIG. 17 includes a plurality of identifiers 244 that are visibly recognizable. The visual identifiers 244 visually distinguish the desired zone 218 from the areas adjacent to the desired zone 218. Setting off the desired zone 218 from the areas adjacent to the desired zone 218 allows the medical professional to place the sleeve 200 correctly into the port site 40. In this illustrated embodiment, the visual identifiers 244 are located on the outer surface 234 of the expandable fluid delivery sleeve 200, however, those skilled in the art recognize that the visual identifiers 244 could be located upon any fluid delivery member, including, but not limited to, the expandable fluid delivery sleeve 200, the needle 232, the trocar and cannula assembly 236, or any other fluid delivery member usable for visualization purposes during the fluid delivery procedure. In some embodiments, the visual identifier(s) 244 may be one or more different colors. In other embodiments, the visual identifiers 244 are markings made upon the expandable fluid delivery sleeve 200. Other embodiments use stripes, dots, or even shades of the same color. Moreover, in some alternative embodiments, small raised bumps or other physical constructions are used as visual identifiers 244. Those skilled in the art will recognize that the visual identifiers 244 can vary between different embodiments so long as they visually distinguish the desired zone 218 from the adjacent areas.

In this embodiment, the visual identifiers 244 are three different colors. Those skilled in the art recognize, however, that only one color would be necessary to distinguish the desired zone 218 from the adjacent areas. The first one is a red/pink area 246 to describe the area just above the desired zone 218 containing the perforations 216. The other visual identifier 244 is the blue colored area 248 to indicate the location of the desired zone 218 containing the perforations 216. The yellow/gold area 250 indicates the area right below the desired zone 218 containing the perforations 216.

During introduction of the expandable fluid delivery sleeve 200 into the port site 40, the medical professional will observe the color coded zones in the port site or the patient's body cavity (e.g., the abdominal cavity or a joint cavity, etc.). This enables one skilled in the art of laparoscopy or arthroscopy to specifically identify where the zone of fluid delivery or infusion is located relative to the anatomy. This visual clue enables accurate fluid delivery via the expandable sleeve of a biologically active substance into the appropriate area. In this illustrative embodiment, if a portion of the blue area 248 is visible, whether from outside the patient through the naked eye, or by an endoscope inside the body cavity, the medical professional can adjust the position of the expandable fluid delivery sleeve 200 to ensure the fluid is delivered directly to the port site 40. Accordingly, use of the visual identifiers 244 assists the medical professional in precisely placing the expandable fluid delivery sleeve 200 into the port site 40.

Moreover, the lengths of these identifiers 244 vary in alternate embodiments depending on the intended patient. For example, a patient with a higher body fat percentage may require a longer desired zone 218 therefore requiring a longer blue area 248. The other visual identifiers 244 above and below the desired zone 218 may also need to be changed in length. Conversely, a patient with a low body fat content or a pediatric patient may need a shorter desired zone and accordingly, the length of the visual identifiers 244 would be different.

Many different types of irrigation fluids may be introduced through the fluid delivery devices of this invention. These include, but are not limited to, saline solutions, lidocaine-containing fluids, betadine-containing fluids, cancer treatment fluids, or any other fluid necessary or desired for a particular medical procedure. In addition, fluids other than irrigation fluids or treatment fluids may be delivered through the devices of this invention. As one additional example, bioadhesives may be delivered to an incision site or any other necessary tissue repair site to provide for quicker and more effective administration of the adhesive to the desired site. Many different types of trocars and cannulas may be utilized within the scope of this invention. These trocars and cannulas may be inserted through a port site of a patient together in one operation or separately, for example, by using a needle introducer for an expandable cannula and subsequently introducing the trocar and cannula assembly as illustrated.

While the present invention has been illustrated by a description of a preferred embodiment and while this embodiment has been described in some detail, it is not the intention of the Applicant to restrict or in any way limit the scope of the appended claims to such detail. Devices of this invention may be used in many different surgical fields including, but not limited to, the fields of arthroscopic and laparoscopic surgery. Additional advantages and modifications will readily appear to those skilled in the art. The various features of the invention may be used alone or in numerous combinations depending on the needs and preferences of the user. This has been a description of the present invention, along with the preferred methods of practicing the present invention as currently known. However, the invention itself should only be defined by the appended claims, wherein I claim:

1. A method for administering fluid directly to a port site located in a section of tissue, the method comprising:
a) introducing a radially expandable tubular structure into said port site, said radially expandable tubular structure having an outer surface adapted to interface with said port site and defining a lumen therethrough; and
b) delivering said fluid to said port site via at least one fluid passageway in fluid communication with said outer surface, said at least one fluid passageway including a portion at least defined in part by said radially expandable tubular structure.

2. A method for administering fluid directly to a port site located in a section of tissue, the method comprising:
a) placing an insert into a lumen defined in a radially expandable tubular structure having an outer surface adapted to interface with said port site;
b) introducing said radially expandable tubular structure and insert into said port site; and
c) delivering said fluid to said port site via at least one fluid passageway in fluid communication with said outer surface, said at least one fluid passageway including a portion at least defined in part by said radially expandable tubular structure.

3. The method of claim 2, further including expanding said radially expandable tubular structure.

4. The method of claim 2, further including:
d) removing said insert; and
e) inserting a second insert into said lumen.

5. The method of claim 4, wherein said insert is a needle and said second insert is a trocar and cannula assembly.

6. The method of claim 5, wherein said trocar and cannula assembly dilates said radially expandable tubular sleeve.

7. The method of claim 2, wherein said insert is a needle.

8. The method of claim 2, wherein said portion includes an area defined between the lumen and the insert.

9. The method of claim 2, wherein said at least one fluid passageway includes a lumen defined in said insert.

10. The method of claim 2, wherein said portion includes a channel defined between the outer surface of said radially expandable tubular structure and the lumen of said radially expandable tubular structure.

11. An apparatus to administer fluid into a port site formed in an area of tissue from a location outside of said port site via at least one fluid passageway, the apparatus comprising:
a radially expandable tubular structure defining a lumen therethrough, said radially expandable tubular structure having an outer surface constructed and arranged to interface with tissue, wherein said at least one fluid passageway is in fluid communication with said outer surface, wherein said at least one fluid passageway includes a portion at least defined in part by said radially expandable tubular structure; and
an insert passing into said lumen.

12. The apparatus of claim 11, wherein said insert is a needle.

13. The apparatus of claim 11, wherein said insert is a trocar and cannula assembly.

14. The apparatus of claim 11, wherein said at least one fluid passageway includes a lumen defined through said insert and perforations formed through at least said outer surface.

15. The apparatus of claim 11, wherein said at least one fluid passageway includes the area located between said insert and said lumen and perforations formed through at least said outer surface.

16. The apparatus of claim 11, wherein said at least one fluid passageway includes a channel defined in said radially expandable tubular structure and perforations formed through at least said outer surface.

17. The apparatus of claim 11, wherein said outer surface of said radially expandable tubular structure includes a visual identifier, on said outer surface, said visual identifier visually distinguishing the location of said fluid passageway relative to an adjacent area of said outer surface.

18. The apparatus of claim 17, wherein said visual identifier comprises a color.

19. The apparatus of claim 17, wherein said visual identifier identifies a portion of said outer surface of said radially expandable tubular structure defining a plurality of perforations in communication with said fluid passageway.

20. A kit for administering fluid into a port site formed in a section of tissue from a location outside of said port site comprising:
a radially expandable tubular structure defining a lumen therethrough, said radially expandable tubular structure having an outer surface adapted to interface with said port site, said radially expandable tubular structure having a distal end;
a needle for insertion into said lumen, said needle assisting with implantation of said radially expandable tubular structure; and
a cannula having an external diameter greater than the diameter of said lumen, wherein insertion of said cannula into said lumen radially expands said radially expandable tubular structure;
wherein at least one fluid pathway is in fluid communication with said location outside of said port site and at least one of said outer surface and said distal end.

21. A method for directing the application of fluid into a port site formed in a patient, comprising:
introducing a fluid delivery member into said port site, said fluid delivery member defining at least one fluid passageway in fluid communication with said port site; and
observing a visual identifier on said fluid delivery member to visually distinguish the location of the at least one fluid passageway relative to an adjacent area of said fluid delivery member.
